(19) United States
(12) Patent Application Publication
(10) Pub. No.: US 2021/0068938 A1
(43) Pub. Date: Mar. 11, 2021

(54) MULTIPURPOSE IMPLANT WITH MODELED SURFACE STRUCTURE

(71) Applicants: Viacheslav Agarkov, Aliso Viejo, CA (US); Igor Uspenskiy, Nizhniy Novgorod (RU); Yury Kolmogorov, Nizhniy Novgorod (RU)

(72) Inventors: Viacheslav Agarkov, Aliso Viejo, CA (US); Igor Uspenskiy, Nizhniy Novgorod (RU); Yury Kolmogorov, Nizhniy Novgorod (RU)

(21) Appl. No.: 16/820,692

(22) Filed: Mar. 16, 2020

Related U.S. Application Data
(63) Continuation-in-part of application No. 15/339,805, filed on Oct. 31, 2016, now Pat. No. 10,588,732.
Application No. 15/339,805 is a continuation-in-part of application No. 14/323,891, filed on Jul. 3, 2014, now Pat. No. 9,480,777.
(60) Provisional application No. 61/979,895, filed on Apr. 15, 2014.

(30) Foreign Application Priority Data
Mar. 7, 2014 (RU) 2014108943

(51) Int. Cl. A61F 2/00; A61F 2/30; A61L 27/26; A61L 27/56

(52) U.S. Cl. CPC A61F 2/0063; A61L 2430/34; A61F 2/0077; A61F 2/30771; A61L 27/26; A61L 27/56; A61F 2250/0087; A61F 2230/0008; A61F 2230/0019; A61F 2230/0006; A61F 2002/3071; A61F 2002/30143; A61F 2002/30153; A61F 2002/30113; A61F 2002/30125; A61F 2002/0068; A61F 2002/0081; A61F 2002/30772; A61F 2002/30069; A61L 2430/06; A61F 2/30756

(57) ABSTRACT

Embodiments of an implant for use in surgery are disclosed. The implant may include elastic polymer file made from a suitable biologically compatible polymer. The implant may also include a reinforcement element.

### CROSS-REFERENCE TO RELATED APPLICATIONS

This application claims priority as a continuation-in-part of U.S. application Ser. No. 15/339,805, filed Oct. 31, 2016, now U.S. Pat. No. 10,588,732, which claims priority as a continuation-in-part of U.S. application Ser. No. 14/323,891, filed Jul. 3, 2014, now U.S. Pat. No. 9,480,777, which claims priority to Russian Patent Application No. 2014108943, filed on Mar. 7, 2014, and U.S. Provisional Patent Application No. 61/979,895, filed on Apr. 15, 2014, each of which is incorporated by reference in their entirety.

### BACKGROUND

Modern methods of soft tissue reconstruction call for the simultaneous use of materials that frequently have several incompatible properties. For example, in treating ventral hernia through the intra-peritoneal on-lay mesh method (laparoscopic IPOM), the synthetic implant material should ensure anti-adhesion on the visceral side (facing the internal organs). On the parietal side (facing the abdominal wall) it is desirable to ensure the tissue's controllable integration into the implant. The growing tissues should not shrink or crimple the implant in the distant post-operation period. At the same time, the tissue integration should reliably secure it to the abdominal wall tissue.

The porous structure of the implant surface should also meet criteria. For instance, macrophage cells and neutrophiles, killers of bacteria, are unable to penetrate fine pores measuring less than 10 μm. This enables the bacteria, smaller than 1 μm, to form colonies in pores measuring less than 10 μm and in spaces of multi-filament meshes, which causes a risk of infection. Therefore it is desirable for the implant to have a structure in which the pores and gaps in the mesh plexus nodes would not be below 75 μm. See C N Brown, J G Finch “Which mesh for hernia repair?”, Ann R Coll Surg Engl. 2010 May, available at http://www.ncbi.nlm.nih.gov/pmc/articles/PMC3025220/. It is desirable that the synthetic implant should have a minimum tissue response and be strong and elastic enough for clinical applications. It is desirable that the implant should be able to be sutured or fastened with a surgical stapler. The strength of the implant should be commensurable to the stress sustained by the abdominal wall during coughing, jumping, etc. (e.g., tensile strength up to 32 N/cm). At the same time, the implant should feature elasticity close to that of the abdominal wall (e.g., up to 38% at the maximum stress).

The task of creating such an implant has not been fulfilled since the implants known to date do not provide all of the desired capabilities.

Currently available implants contain layers of different non-absorbable materials fastened together by some means. In most cases, the layer that ensures integration of the abdominal wall tissues is a polypropylene or polyester mesh whilst the layer that provides the anti-adhesive barrier is made from polytetrafluoroethylene or, for instance, collagen. Such designs are described in the following patents and publications: U.S. Pat. No. 6,258,124 titled “Prosthetic repair fabric”, U.S. Pat. No. 6,652,595 titled “Method of repairing inguinal hernias”, U.S. Pat. No. 5,743,917 titled “Prosthesis for the repair of soft tissue defects”, U.S. Patent Publication No. 20020052654 titled “Prosthetic repair fabric”, U.S. Pat. No. 8,206,632 titled “Method of making composite prosthetic devices having improved bond strength”, and U.S. Pat. No. 8,623,096 titled “Double layer surgical prosthesis to repair soft tissue,” the entirety of each is hereby incorporated by reference.

Implants are available that are essentially in the form of a mesh from a stable strong material (polypropylene, polyester or other) coated with a temporary absorbable anti-adhesive material. The mesh is designed for soft tissues to grow into it whilst the absorbable layer, separating the mesh from visceral tissues, creates a temporary anti-adhesive barrier promoting the formation of peritoneum and minimizing the probability of union with the mesh during the wound healing. Following the biological degradation of the barrier, the mesh integrates into the abdominal wall tissue.

Such designs are described in the following publications: U.S. Patent Publication No. 20130317527 titled “Single plane tissue repair patch having a locating structure”, U.S. Patent Publication No. 20130267971 titled “Single plane tissue repair patch”, and U.S. Patent Publication No. 20130267970 titled “Single plane tissue repair patch,” the entirety of each is hereby incorporated by reference. An example of commercial use of such a design is an implant under the trade name of PHYSIOMESH manufactured by ETHICON, Inc.

All these implants feature strength that ensures a high restorative effect and are fit for suture-aided fixation, but are disadvantageous in some aspects.

By virtue of its micro-porous structure, polytetrafluoroethylene mollifies the gravity and reduces the commissural side effects of the healing process, but does not altogether eliminate them. The use of collagen implies a high risk of a tissue rejection, allergic response or infection.

Another disadvantage is the shrinkage of the implant, which is specific to materials known to date (polypropylene, polyester). Growing through the mesh, the organism tissues contribute to its extra shrinkage and wrinkling, which negatively impacts the quality of the patient's life.

It is not recommended to introduce implants coated with a temporary absorbable anti-adhesive material in the event of a casual or scheduled opening of the digestive tract lumen or in the event of infection of the site since this may result in the infection of the implant itself, as its absorbable material promotes colonization of microorganisms, which may trigger a post-operative pyoinflammatory process.

These implants have either a mesh-like or porous structure, which ensures integration of the abdominal wall tissues, but makes it impossible to control the size of the mesh pore and cell. The material structure is usually determined by the range of pore and cell size. In woven materials the mesh weave areas are inaccessible during sterilization and are potentially a place of microbial contamination and a site of bacterial infection.

All these factors may restrict the use of implants in various clinical cases.

### SUMMARY

For purposes of summarizing the disclosure, certain aspects, advantages and novel features of the disclosure have been described herein. It is to be understood that not necessarily all such advantages can be achieved in accordance with any particular embodiment disclosed herein. Thus, the embodiments disclosed herein can be embodied or carried out in a manner that achieves or optimizes one advantage or group of advantages as taught herein without necessarily achieving other advantages as can be taught or suggested herein.

The aim of certain embodiments of this invention is to provide a new implant, method of manufacture and method of use that addresses, reduces, or eliminates one or more of the above said disadvantages and/or fulfills one or more of the desired capabilities mentioned above.

The offered implant is intended for surgery ventral hernias by the laparoscopic IPOM method (intra-peritoneal onlay mesh).

A surgical implant for reconstruction of soft tissues comprising: an elastic film formed from a non-porous biologically compatible cross-linked copolymer based on 50-75 percent by weight multi-functional urethane (meth)acrylate oligomers and 20-50 percent by weight methacrylate monomers; the elastic film having: a first smooth anti-adhesive surface, and a second relief surface opposite the first surface, comprises indentations; wherein the elastic film is processed by soaking in isopropyl alcohol so as to block residual free radicals, thereby decreasing a risk of tissue reaction; and a reinforcement element in the form woven synthetic mesh enclosed inside the elastic film, wherein the reinforcement element covered with the film across the entire first smooth anti-adhesive surface area of the implant and integrated with film such that portions of the reinforcement element are exposed through the indentations of the second relief surface.

The first smooth surface has a roughness that does not exceed about 50 nanometers.

In some embodiments, the reinforcement element may be polypropylene mesh or polyester mesh, and of different thickness (for instance, 20-50 microns).

The elastic film has the oval shape or the shape of a rectangle with rounded corners.

In some embodiments, the elastic film is continuous without holes or apertures such that the reinforcement element is covered with the film across the entire first smooth anti-adhesive surface area of the implant.

In some embodiments, the elastic film comprises holes through the film in the area of the indentations of the second relief surface. The holes have the diameter 0.5-2.0 mm. These holes serve to reduce grey formation and to eliminate the need to drain cavities with seromas. The holes prevent the formation of clusters of serous fluid that can naturally seep through them into the abdominal cavity.

The size of holes allows serous fluid, which remains in the tissues after closure, to seep through them, but the size of holes is too small to allow adjacent tissues to grow through them from the side of the first smooth surface of the film. Thus, the organs and tissues that contact with the first smooth surface slide on it without forming any synechias.

In some embodiments, the elastic film comprises an inscription for indication of film surfaces. During the operation, it is difficult for the surgeon to determine to the touch of the implant surface due to gloves. For this reason, an inscription is made on the polymer film. Mainly polymer elastic film of the implant is made transparent. The inscription is formed by a polymeric material, stained in color, and is enclosed inside the polymer film. The inscription is mainly made of the same material as the polymer elastic film, in which a pigment is added, for example a blue one. For example, the inscription is “visceral side”, which is correctly read from the side of a smooth anti-adhesion surface. Alternately, the inscription may be made in the form of a relief consisting of letters from the side of the second relief surface.

In some embodiments, the implant comprises openings with pre-installed polypropylene filaments for fixation of the implant. The implant contains pre-installed polypropylene filaments on the periphery to fix the implant during surgery. The fixation site of the polypropylene filament is a recess on the film from the side of a relief surface, through which a synthetic fabric mesh is exposed. Polypropylene filaments are inserted through the nettings of the synthetic mesh and tied into the knot from the side of the second relief surface. Thus, fixation polypropylene filaments don't contact with the first smooth antiadhesive surface. Around the site, where the polypropylene filament is attached, the relief of the second surface is made in such a way that the reinforcement of the fastening of the synthetic fabric mesh is formed, for example, the texture is made in the form of a wide polymer ring. Fixation reinforcement is necessary to prevent the synthetic fabric mesh from detachment/tearing off the polymer film during a strong tension of the implant by the surgeon. The fixation filaments may be removed after fixation with a hernia stapler. Removal of filaments reduces the likelihood of chronic pain, in the near and distant postoperative period. Threads can be left if special reliability fixation (complex postoperative hernias) is required. Leaving or removing the polypropylene filaments is the decision of the operating surgeon.

In some embodiments, the surface structure of the spatially sewn or sutured polymer is not porous and is preset during manufacture. Since elastic film with smooth surface is an anti-adhesive barrier, then a high level of smoothness is set during manufacture. For example, the surface roughness may not exceed about 50 nanometers, such as be between about 5 and about 20 nanometers. At least the smooth surface can be nonporous so as to prevent or minimize the risk of creating undesired tissue formations (or spikes). The surface of an implant can be inert in order to decrease the reaction of the tissues to the implant. In order to achieve this, the polymer may undergo additional procedure of blocking of free radicals, for example by means of processing of a surface of isopropyl alcohol. Such a level of smoothness prevents the commissure formation (e.g., tunica growth and adhesion, radicular-muscular accretion, etc.) and enables the tissues, contacting with this surface, to move and slide freely.

The second relief surface opposite the first surface, comprises indentations and the reinforcement element integrated with film such that portions of the reinforcement element are exposed through the indentations of the second relief surface. The reinforcement element is non-degradable. The second relief surface opposite the first surface, comprises indentations, has a form of mesh with cells. In the post-operative period the adjacent tissues can grow into the cells of this embossed pattern without penetrating the polymer. Thus, in some embodiments, in the post-operative period the tissues that have grown into the tissue cells are unable to shrink, wrinkle, or destroy the implant. Synthetic woven mesh acts as the reinforcing element and can be various thickness (for example, 20-50 microns) and is made of polypropylene or polyester. Presence of reinforcing element provides possibility of suturing or fastening by surgical stepper of endoprosthesis.

The production process can exclude the generation of free radicals, which minimizes the triggering of a tissue response.

In some embodiments, the implant can be manufactured by polymerization in molds, consisting of two flat surfaces. The manufacturing process may use any available polymerization method, such as photopolymerization, thermal polymerization and others. As an example, disclosed embodiments are an improvement over the embodiments described in European Patent Publication No. EP 2644348 titled “A method of manufacturing an artificial elastic implant for restorative and reconstructive surgery,” which is incorporated by reference in its entirety. Embodiments of the implants, methods and other features described in EP 2644348 may also be applied to embodiments described in this application.

In some embodiments, a surgical implant includes a non-degradable reinforcement element at least partially enclosed in a polymer film, the film including spatially linked polymer obtained by photopolymerization of methacrylic oligomers and monomers. Polymerization can be thermal polymerization.

### BRIEF DESCRIPTION OF THE DRAWINGS

Embodiments of the present application will now be described hereinafter, by way of example only, with reference to the accompanying drawings in which:

FIGS. 1A-1C illustrate a top view, a profile view and a perspective view, respectively, of an implant according to some embodiments.

FIGS. 2A-2C illustrate a top view, a profile view and a perspective view, respectively, of another implant according to some embodiments.

FIGS. 3A-3C illustrate a top view, a profile view and a perspective view, respectively, of another implant according to some embodiments.

FIGS. 4A-4C illustrate a top view, a profile view and a perspective view, respectively, of another implant according to some embodiments.

FIGS. 5A-5C illustrate a top view, a profile view and a perspective view, respectively, of another implant according to some embodiments.

FIGS. 6A-6C illustrate a top view, a profile view and a perspective view, respectively, of another implant according to some embodiments.

FIGS. 7A-7B illustrate a top view, a profile view and a perspective view, respectively, of another implant according to some embodiments.

FIGS. 8A-8B illustrate a top view, a profile view and a perspective view, respectively, of another implant according to some embodiments.

FIGS. 9A-9B illustrate a top view, a profile view and a perspective view, respectively, of another implant according to some embodiments.

FIGS. 10A-10B illustrate a top view, a profile view and a perspective view, respectively, of another implant according to some embodiments.

FIGS. 11A-11B illustrate a top view, a profile view and a perspective view, respectively, of another implant according to some embodiments.

FIGS. 12A-12F illustrate exemplary shapes and forms of texture of the outer surface of the polymer film.

FIGS. 13A-13C illustrate a top view, a profile view and a perspective view, respectively, of another implant according to some embodiments.

FIGS. 14A-14F illustrate a top view, a bottom view and a profile view of an implant according to some embodiments.

FIGS. 15A-15F illustrate a top view, a bottom view and a profile view of an implant according to some embodiments.

FIGS. 16A-16B illustrate a top view and a bottom view of an implant according to some embodiments.

FIGS. 16C-16D illustrate a top view and a bottom view of an implant according to some embodiments.

### DETAILED DESCRIPTION

While certain embodiments are described, these embodiments are presented by way of example only, and are not intended to limit the scope of protection. Indeed, the novel methods and systems described herein may be embodied in a variety of other forms. Furthermore, various omissions, substitutions, and changes in the form of the methods and systems described herein may be made without departing from the scope of protection.

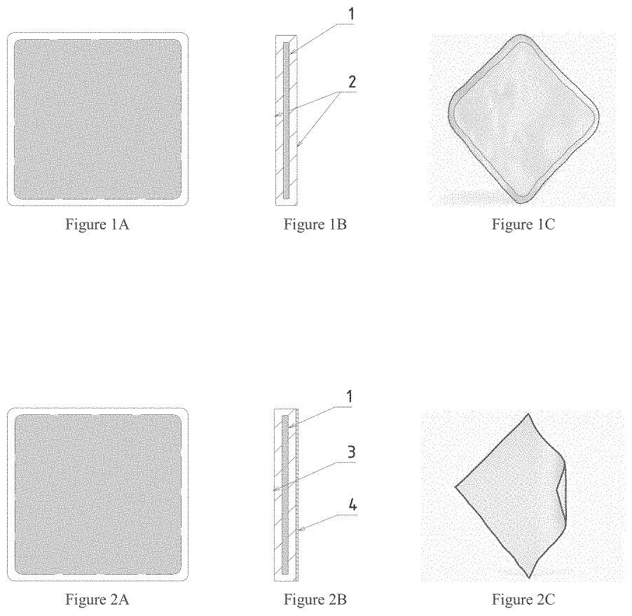

FIGS. 1A-1C illustrate an implant according to some embodiments. The implant of FIGS. 1A-1C is illustrated having a square shape, but it will be appreciated that this and other implants may have any desired shape. The illustrated implant has a profile section with a reinforcement element (1) covered with the film across the entire surface area of the element. The illustrated implant has both surfaces (2) that are smooth. The drawing in the FIG. 1A schematically illustrates the implant, while the drawing in FIG. 1C depicts a manufactured implant.

FIGS. 2A-2C illustrate an implant according to some embodiments. The illustrated implant has a profile section with a reinforcement element (1) covered with the film across the entire surface area of the element. The illustrated implant has one surface (3) that is smooth, while the other surface (4) is textured or embossed. The smooth surface (3) can be anti-adhesive so as to minimize tissue adhesion, while the textured surface can promote adhesion and integration into the tissue. The drawing in FIG. 2A schematically illustrates the implant, while the drawing in FIG. 2C depicts a manufactured implant.

The embossed surface (4) can include a pattern of a preset size, depth and cell (or pore) shape. For example, the pattern can include cells measuring about 75 μm (microns) by about 75 μm and be about 50 μm deep. As another example, the cells can measure about 75 μm in diameter and be about 50 μm deep. The cells can have circular, rectangular, hexagonal, or any other suitable shape and can be of any suitable size. The pattern may include cells of more than size and shape. For example, the embossed pattern can be in the form of a mesh, numbers, letters or their combination. The embossed pattern can be regular (e.g., not open-ended). The embossed surface can facilitate fixation to adjacent tissue.

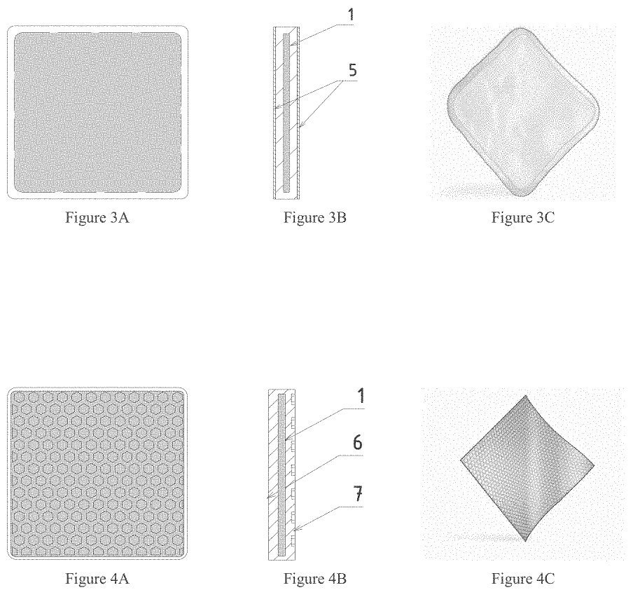

FIGS. 3A-3C illustrate an implant according to some embodiments. The illustrated implant has a profile section with a reinforcement element (1) covered with the film across the entire surface area of the element. The illustrated implant has both surfaces (5) that are textured or embossed. The drawing in FIG. 3A schematically illustrates the implant, while the drawing in FIG. 3C depicts a manufactured implant.

FIGS. 4A-4C illustrate an implant according to some embodiments. The illustrated implant has a profile section with a reinforcement element (1) covered with the film across the entire surface area of the element. The illustrated implant has one surface (6) that is smooth, while the other surface (7) is textured or embossed. The pattern of the textured surface is a pattern of hexagons regularly repeated over the entire surface. The drawing in FIG. 4A schematically illustrates the implant, while the drawing in FIG. 4C depicts a manufactured implant. The textured surface (7) is illustrated by the drawings in FIGS. 4A and 4C.

In some embodiments, the reinforcement element can cover or be embedded in less than the entire surface area of the implant. For example, the implant can include one or more reinforcement element sections. Sections of the reinforcement element can have any suitable shape, such as square, rectangular, circular and radial strip shape. In some embodiments, sections of the reinforcement element can be covered with film on both sides, with the film covering not only synthetic material but also portions extending between sections of the reinforcement element. The film may have the same texture as sections of the implant that do not include the reinforcement element inside the film. In other embodiments, sections of the reinforcement element can be covered with film having different smoothness or texture as sections of the implant that do not include the reinforcement element inside the film. For example, sections of the reinforcement element can be covered with smooth film while other sections of the implant that do not include the reinforcement element have textured film.

FIGS. 5A-5C illustrate an implant according to some embodiments. The illustrated implant has a profile section with a reinforcement element (1) not covering or being embedded within the entire surface area of the implant. In the illustrated implant, the reinforcement element forms radial rays (or strips) extending from the center of the circle, and the reinforcement element also extends along the periphery of the circle. The reinforcement element can be covered by a polymer film (illustrated as having a circular shape) on both sides. The polymer film may have the same or different texture than the texture of the film in the sections (illustrated as sectors) not having the reinforcement element inside the film. For example, the reinforcement element can be covered with smooth film while other sections having no underlying reinforcement element may have textured film (e.g., such sections may have partially mesh-like texture). The reinforcement element can be cut into desired shapes (e.g., strips and circle) using laser cutting. The drawing in FIG. 5A schematically illustrates the implant, while the drawing in FIG. 5C depicts a manufactured implant. The drawing in FIG. 5C illustrates the textured and smooth surfaces of the implant.

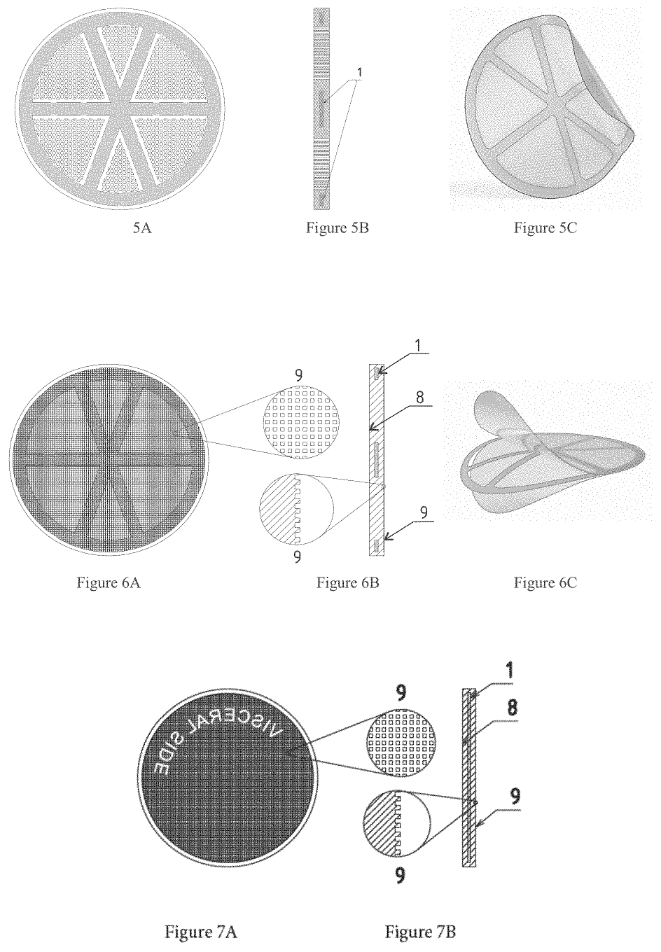

FIGS. 6A-6C illustrates an implant according to some embodiments. The illustrated implant has a profile section with a surface area of a reinforcement element (1) being smaller than the surface area of the implant. In the illustrated implant, the reinforcement element forms radial rays (or strips) extending from the center of the circle, and the reinforcement element also extends along the periphery of the circle (e.g—extends circumferentially). The reinforcement element can be covered by a polymer film (illustrated as having a circular shape) on both sides. The film on opposite sides may have the same or different characteristics. One surface (8) of the implant on one side of the reinforcement element can be smooth, while the other surface (9) on the other side of the reinforcement element can be textured or embossed. The drawing in FIG. 6A schematically illustrates the implant, while the drawing in FIG. 6C depicts a manufactured implant. The drawing in FIG. 6C illustrates a cross-sectional view of the implant and depicts the reinforcement element having radial rays sections extending from the center and a section extending along the periphery of the implant.

FIGS. 7A-7B illustrates an implant according to some embodiments. The illustrated implant has a profile section with a surface area of a reinforcement element (1) covered with the film across the entire surface area of the element. The reinforcement element can be covered by a polymer film (illustrated as having a circular shape) on both sides. The film on opposite sides may have the same or different characteristics. One surface (8) of the implant on one side of the reinforcement element can be smooth, while the other surface (9) on the other side of the reinforcement element can be textured or embossed. The reinforcement element (1) may be solid across a surface area, without holes or apertures, as shown in FIG. 7A, or may have holes as shown in FIG. 6A.

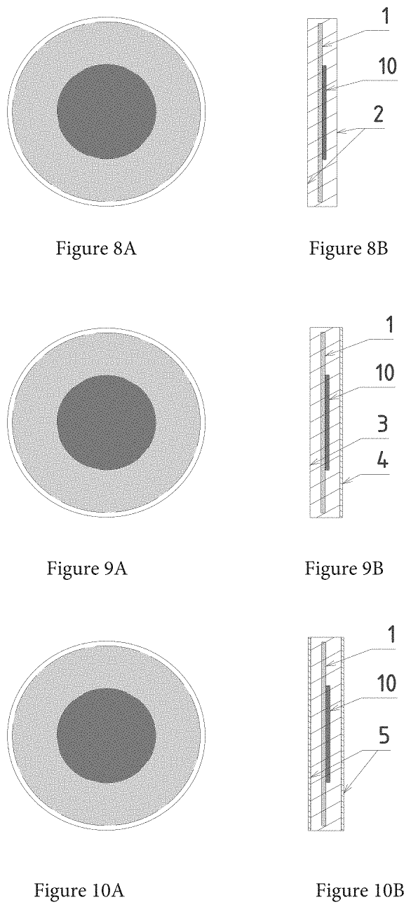

FIGS. 8A-8B illustrates an implant according to some embodiments. The illustrated implant has a profile section with a surface area of a reinforcement element comprising of first reinforcement element (1) and second reinforcement element (10) covered with the film across the entire surface area of the element. The reinforcement element can be covered by a polymer film (illustrated as having a circular shape) on both sides. The illustrated implant has both surfaces (2) that are smooth. The second reinforcement element (10) may be the same size, or smaller than the first reinforcement element.

FIGS. 9A-9B illustrates an implant according to some embodiments. The illustrated implant has a profile section with a surface area of a reinforcement element comprising of first reinforcement element (1) and second reinforcement element (10) covered with the film across the entire surface area of the element. The reinforcement element can be covered by a polymer film (illustrated as having a circular shape) on both sides. The illustrated implant has one surface (3) that is smooth, while the other surface (4) is textured or embossed. The smooth surface (3) can be anti-adhesive so as to minimize tissue adhesion, while the textured surface can promote adhesion and integration into the tissue. The second reinforcement element and the textured surface may be on the same side of the first reinforcement element, and on an opposite side of the first reinforcement element than the smooth side.

FIGS. 10A-10B illustrates an implant according to some embodiments. The illustrated implant has a profile section with a surface area of a reinforcement element comprising of first reinforcement element (1) and second reinforcement element (10) covered with the film across the entire surface area of the element. The reinforcement element can be covered by a polymer film (illustrated as having a circular shape) on both sides. The illustrated implant has both surfaces (5) that are textured or embossed.

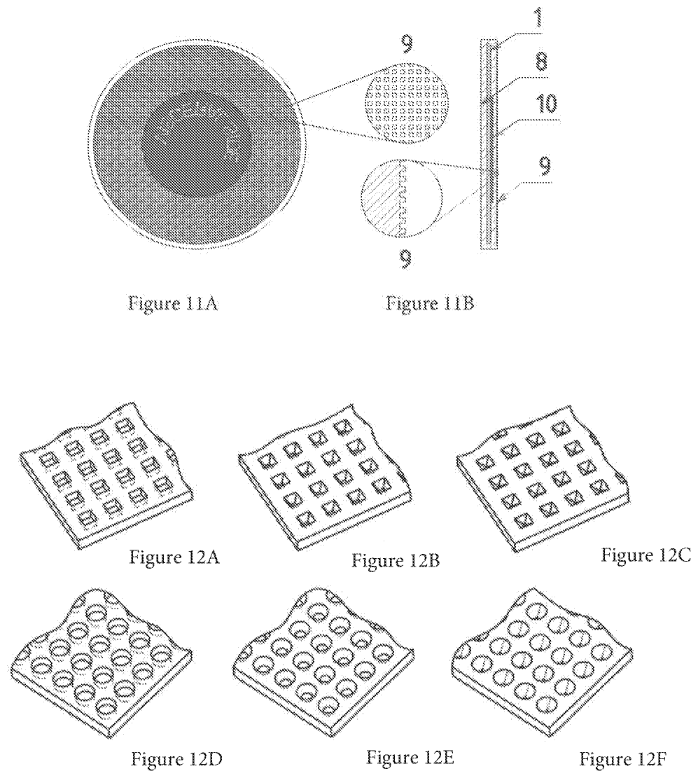

FIGS. 11A-11B illustrates an implant according to some embodiments. The illustrated implant has a profile section with a surface area of a reinforcement element comprising of first reinforcement element (1) and second reinforcement element with cutouts in the form of text (10) covered with the film across the entire surface area of the element. The reinforcement element can be covered by a polymer film (illustrated as having a circular shape) on both sides. The film on opposite sides may have the same or different characteristics. One surface (8) of the implant on one side of the reinforcement element can be smooth, while the other surface (9) on the other side of the reinforcement element can be textured or embossed.

FIGS. 12A-12F illustrates exemplary shapes and forms of texture of the outer surface of the polymer film. For example, the shapes may include rectangular or cubic indentations, square, rectangular, or polygonal pyramid indentations either coming to a point or being truncated, cylindrical, or conical either coming to a point or being truncated.

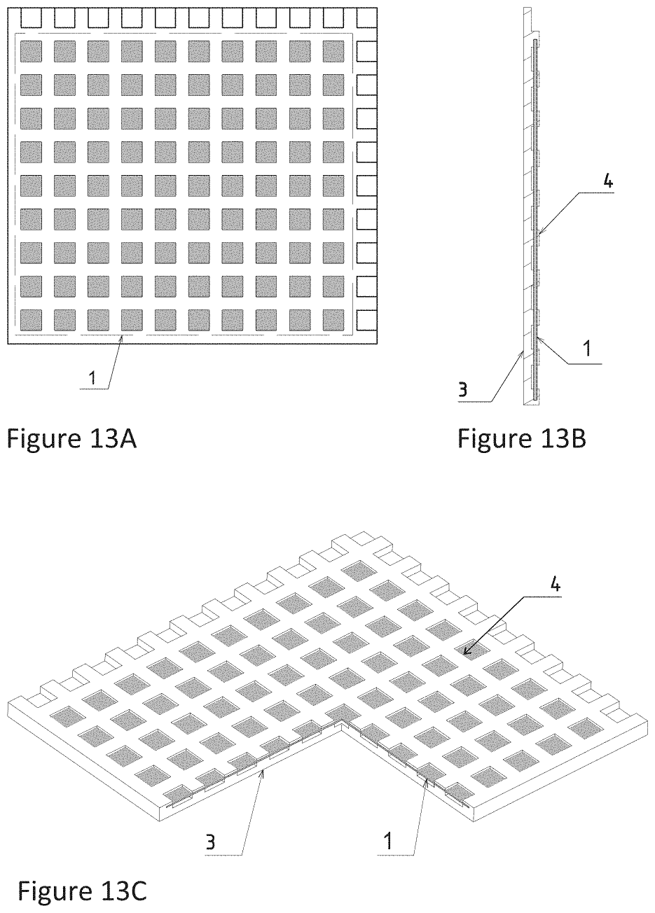

FIGS. 13A-13C illustrate an implant according to some embodiments. The illustrated implant has a top view, profile section with a reinforcement element (1) covered with the film across the entire anti-adhesive surface area of the one side of implant. The illustrated implant has one surface (3) that is smooth, while the other surface (4) is textured or embossed. The smooth surface (3) can be anti-adhesive so as to minimize tissue adhesion, while the textured surface can promote adhesion and integration into the tissue. The drawing in FIG. 13A-13B schematically illustrates the implant, while the drawing in FIG. 13C depicts a view of textured surface (4).

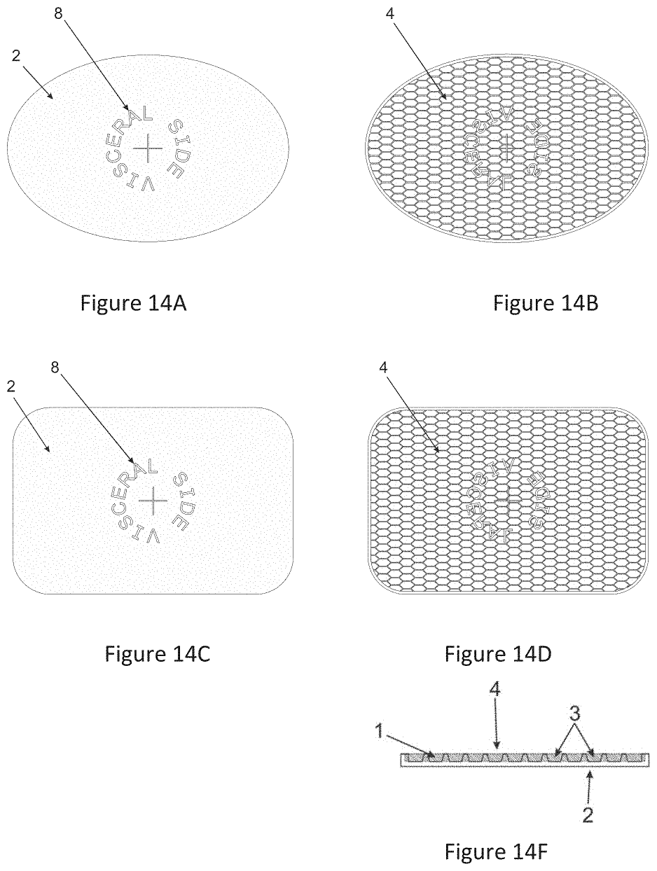

FIGS. 14A-14F illustrate an implant according to some embodiments. FIG. 14A, 1C illustrated implant from a first smooth anti-adhesive surface. FIG. 14B, 14D illustrated implant from a second relief surface opposite the first surface, comprises indentations. The implant of FIGS. 14A-14B is illustrated having an oval shape. The implant of FIGS. 14C-14D is illustrated having a shape of a rectangle with rounded corners. The FIG. 14F illustrated implant has a profile section elastic film, wherein the reinforcement element (1) covered with the film across the entire first smooth anti-adhesive surface (2) area of the implant and integrated with film such that portions of the reinforcement element (1) are exposed through the indentations (3) of the second relief surface (4).

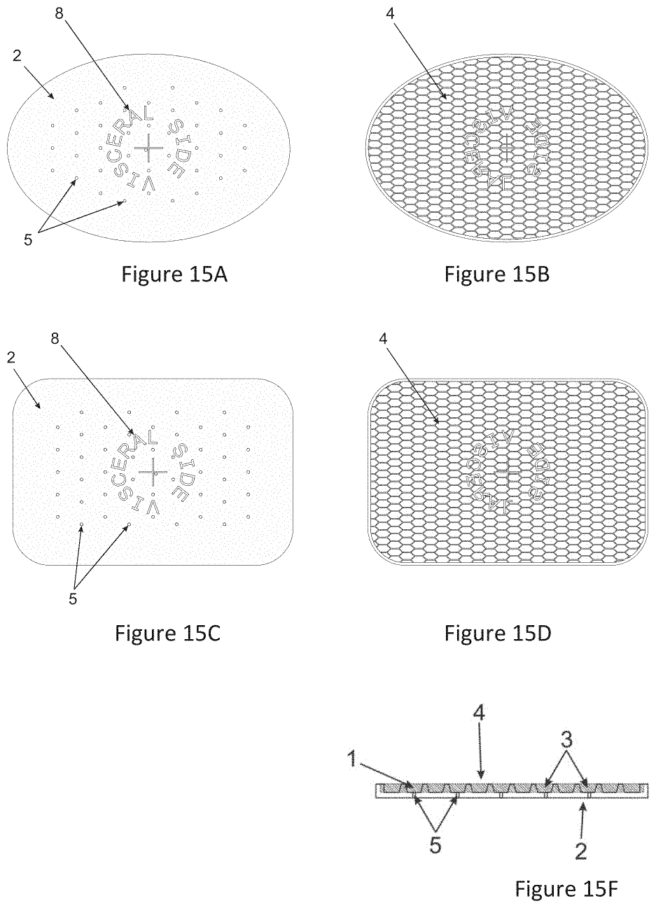

FIGS. 15A-15F illustrate an implant according to some embodiments. FIG. 15A, 2C illustrated implant from a first smooth anti-adhesive surface. FIG. 15B, 15D illustrated implant from a second relief surface opposite the first surface, comprises indentations (3). The implant of FIGS. 15A-15B is illustrated having an oval shape. The implant of FIGS. 15C-15D is illustrated having a shape of a rectangle with rounded corners. The FIG. 15F illustrated implant has a profile section elastic film, wherein the reinforcement element (1) covered with the film across the entire first smooth anti-adhesive surface (2) area of the implant and integrated with film such that portions of the reinforcement element (1) are exposed through the indentations (3) of the second relief surface (4); the elastic film comprises holes (5) through the film in the area of the indentations (3) of the second relief surface (4). The holes (5) have the diameter 0.5-2.0 mm. These holes (5) serve to reduce grey formation and to eliminate the need to drain cavities with seromas.

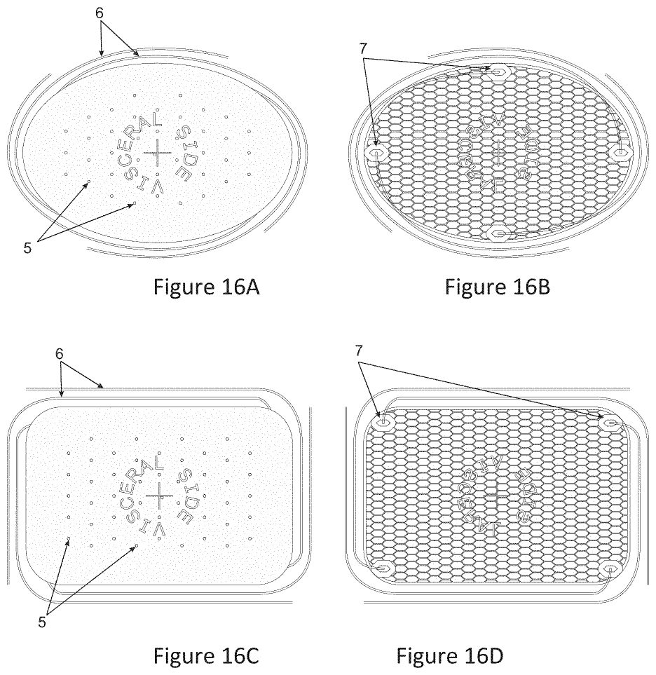

FIGS. 16A-16D illustrate an implant according to some embodiments. FIG. 16A, 16C illustrated implant from a first smooth anti-adhesive surface. FIG. 16B, 16D illustrated implant from a second relief surface opposite the first surface, comprises indentations. The implant of FIGS. 16A-16B is illustrated having an oval shape. The implant of FIGS. 16C-16D is illustrated having a shape of a rectangle with rounded corners. The implant comprises pre-installed polypropylene filaments (6) for fixation of the implant. The fixation site of the polypropylene filament (6) is a recess on the film from the side of a relief surface (4), through which a synthetic woven mesh (1) is exposed. Polypropylene filaments (6) are inserted through the nettings of the synthetic mesh (1) and tied into the knot from the side of the second relief surface (4). Thus, fixation polypropylene filaments (6) don't contact with the first smooth antiadhesive surface (2). Around the site, where the polypropylene filament is attached, the relief of the second surface is made in such a way that the reinforcement of the fastening of the synthetic fabric mesh is formed, for example, the texture is made in the form of a wide polymer ring (7). Fixation reinforcement is necessary to prevent the synthetic woven mesh (1) from detachment/tearing off the polymer film during a strong tension of the implant by the surgeon.

The FIG. 14B, 14D, 15B,15D, 16B, 16D illustrated that second relief surface (4) has indentations (3) with shape of hexagons regularly repeated over the entire surface.

The FIG. 14A-14D, 15A-15D, 16A-16D illustrated that second relief surface (4) has the inscription (8) for indication of the film surfaces. During the operation, it is difficult for the surgeon to determine to the touch of the implant surface due to gloves. For this reason, an inscription is made in the polymer film. Mainly polymer elastic film of the implant is made transparent. The inscription is formed by a polymeric material, stained in color, and is enclosed inside the polymer film. The inscription is mainly made of the same material as the polymer elastic film, in which a pigment is added, for example a blue one. For example, the inscription is “visceral side”, which is correctly read from the side of a smooth anti-adhesion surface. Alternately, the inscription may be made in the form of a relief consisting of letters from the side of the second relief surface.

As shown, the second relief surface (4) may position the indentations (3) through the reinforcement element (1), such that the reinforcement element (1) is not fully enclosed. The indentations (3) may be any shape, such as rectangular, square, circular, ovoid, etc. and may be uniform along the depth or may be tapered. A reinforcement layer (1) is integrated with the elastic film that portions of the reinforcement layer (1) are exposed through the indentations (3) of second relief surface (4). In an exemplary embodiment, both sides of the reinforcement layer (1) are exposed at the indentations (3). The reinforcement layer (1) is traverses through all of the indentations (3).

As shown, the textured surface may position the indentations through the reinforcement element, such that the reinforcement element is not fully enclosed. The textured surface may also permit gaps or space to be formed between the one surface (3) and the reinforcement element (1). For example, the implant may include a solid layer, in which a first side of the solid layer defines a smooth surface and a second side of the solid layer defines a textured surface. The textured surface may comprise indentations. The indentations may be any shape, such as rectangular, square, circular, ovoid, etc. and may be uniform along the depth or may be tapered. A reinforcement layer may be integrated with the solid layer such that portions of the reinforcement layer are exposed through the indentations of the textured surface. In an exemplary embodiment, both sides of the reinforcement layer are exposed at the indentations. The reinforcement layer may traverse through all of the indentations or a portion of the indentations. As shown, a partial perimeter of indentations defining the textured surface may extend around a peripheral edge of the implant, where the partial perimeter of indentations does not include a reinforcing layer. The reinforcing layer may also be textured or provide apertures through the reinforcing layer or may be solid.

In an exemplary embodiment, the reinforcement layer may be a woven, solid, or apertured structure. When the reinforcement layer is exposed through the textured surface, the reinforcement layer may form spaces for tissue ingrowth. For example, the reinforcement layer may provide apertures that permit access to the space between the reinforcement layer and the one surface and other apertures in the reinforcement layer to exit the space between the reinforcement layer and the one surface, thereby creating a member for the tissue to hook to. In an exemplary embodiment, portions of the reinforcement layer may be colored or otherwise provide a pattern that is visible or can be felt through the outer surfaces to orient or align the implant during use.

Exemplary embodiments of the multi-purpose surgical implant are formed from an elastic film formed from a non-porous biologically compatible cross-linked copolymer based on 50-75 percent by weight multi-functional urethane (meth)acrylate oligomers and 20-50 percent by weight methacrylate monomers. The elastic film may have a first smooth anti-adhesive surface, and a second relief surface opposite the first surface. The elastic film may be processed by soaking in isopropyl alcohol so as to block residual free radicals, thereby decreasing a risk of tissue reaction. The surgical implant may also have a reinforcement element partially or fully enclosed inside the elastic film, wherein the reinforcement element does not contact body organs and tissue from the side of the first smooth anti-adhesive surface. The reinforcement layer may comprise a plurality of apertures. In an exemplary embodiment, adjacent apertures and an intermediate reinforcement layer portion connecting the adjacent apertures may be exposed through the elastic film. In an exemplary embodiment, the elastic film forms a continuous surface on one entire side of the reinforcement layer. In an exemplary embodiment, the reinforcement layer is raised above an exposed surface of the elastic film. The reinforcement layer may have protrusions supporting and extending through the reinforcement layer to partially encapsulate a portion of the reinforcement layer and to expose a portion of the reinforcement layer. In an exemplary embodiment, a gap is formed between the reinforcement layer and the elastic film continuous layer.

The invention also relates to a method for manufacturing an artificial elastic implant for restorative and reconstructive surgery, comprising two casting steps performed in a casting mold. The mold has at least a cover (1) that is optically and UV transparent. In a first step, a first layer of a first photo-curable material or of a second photo-curable material is cast while forming a meniscus (4). Using one of two photo masks (5), the mold is irradiated with UV light to cure the first layer. In a second step, a second layer of either the first or the second photo-curable material is cast onto the cured first layer while forming a meniscus. After irradiating the mold again with ultraviolet light, unhardened photo-curable material is re-moved from the product by dissolving in a suitable solvent. After additionally irradiating the product with UV light, the product is soaked, separated from the mold, placed in isopropyl alcohol for 3 to 24 hours and then vacuum dried.

The first photo-curable material is a composition comprising:
25-40 wt.-% benzyl methacrylate
50-70 wt.-% oligourethane methacrylate
1-5 wt.-% methacrylic acid
1-5 wt.-% octyl methacrylate.

The second photo-curable material is a composition comprising:
20-30 wt.-% phenoxyethyl methacrylate
20-30 wt.-% oligourethane methacrylate 1000F
35-45-wt.-% oligourethane methacrylate 5000F
1-5 wt.-% methacrylic acid
1-5 wt.-% ethylene glycol monomethacrylate.

The invention also relates to a method for manufacturing a surgical implant for reconstruction of soft tissues, comprising several casting steps performed in a casting molds. Each mold consists of two plane glasses; at least one of the glass is cover with photomask. The UV irradiation through photomask forms the structure on the product i.e. the size, form, numbers, letters, meshes, shapes corresponding to the embossed pattern on the surface of the formed product. If the layer contains reinforcing material than the material must be placed on the glass with the photomask to be and irradiated through it. After irradiating the mold is to be decoupled, and the photo-curable material may be poured on the previous layer. Then the molding form is to be covered with the glass with the photomask, suitable to form the next layer. After the last irradiating the mold is to be decoupled and unhardened photo-curable material is re-moved from the product by dissolving in a suitable solvent. Suitable solvents include without being limited to, lower alcohols like ethanol, methanol, propanol, i-propanol, ketones like propanone, 4-methyl-pentan-2-one and butanone as well as mixture s of these.

The photo-curable material is a composition comprising:

20-45 wt.-% benzyl methacrylate

50-70 wt.-% oligourethane methacrylate

1-5 wt.-% methacrylic acid

1-5 wt.-% octyl methacrylate.

The oligourethane methacrylate may have the following structure:

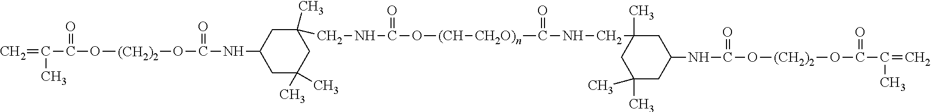

The formulation advantageously contains other ingredients that are common in the field of photocurable materials. These are for example effective amounts of additives capable of initiation of radical polymerization, optical sensitization and/or inhibiting thermal polymerization, dyes or pigments, stabilizers, and the like. Examples are azo-bis-isobutyronitrile, 3,5-di-t-butyl-o-quinone, 2,2-dimethoxyphenyl acetophenone, 2,6-di-tert-butyl-4-metylphenol.

In order to remove all residual monomers, which are left in the cured material and could irritate surrounding tissue after implantation, the polymer is to be irradiated with UV light. Then the polymer must be cured in solvent such as methanol, propanol, i-propanol, propanone, water or the mixture of them at the temperature from 30° C. to 100° C. during 300-1000 min. After drying, the product is ready.

The technical problem to be solved by the invention includes developing a method that allows to produce an implant, which has high elasticity and minimal impact on the surrounding organs and tissues, which has a high biological stability and providing for a reactivity in the post-operative period. The method should also allow producing implants having a uniform surface, either smooth or structured, as well as implants having different partial surfaces, like one smooth and one structured.

This technical problem is solved by a method in accordance with claim 1.

Most of the ingredients used in the method of this invention are commercially available chemicals well-known to the skilled person in the field of polymers. For the oligourethans the following structures apply:

Oligourethane methacrylate 1000F of the following structure:

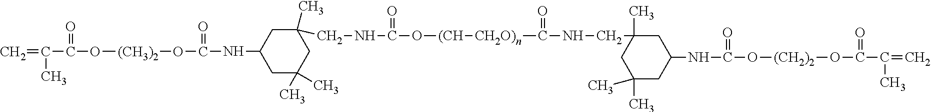

Oligourethane methacrylate 5000F of the following structure:

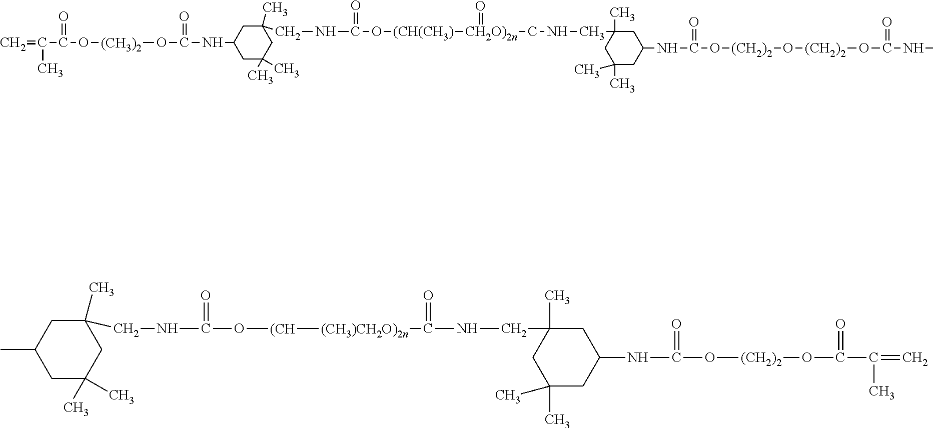

The casting mold, at least the cover, is made from a material which is transparent for visible light as well as UV light in the spectral region needed for photo polymerization. Suitable materials are

UV transparent glass, plastic. The cover can accommodate the photomask into a suitable cavity. The base and the limiting ring can as well be made from other materials like metal, ceramics, plastic. In the present method the mold is not completely filled by the photo polymerizable material. Moreover the material forms a meniscus in its upper region. A meniscus is a free surface of liquid, the shape of which forms under the influence of gravity and the surface energies of the surfaces involved. The surface of the meniscus will remain very smooth during first curing.

It should be noted, that pouring on the material takes place before the limiting ring is being placed on to the base. That means that the amount of photo polymerizable material must be small enough to avoid the material flowing to the rim of the base and from there down.

The photomask is either the first photomask defining the outer geometrical dimensions of the product to be formed, for example a circular, elliptical or square shape, or the second photomask, which on irradiation forms the structure on the surface of the product and is in the form of numbers, letters, meshes, shapes corresponding to the embossed pattern on the surface of the formed product.

After closing the mold it is irradiated with UV light suitable to photo-cure the material. In this first irradiation it is intended to cure the material all the way from top to bottom of the layer. After irradiation the cover and limiting ring are removed and again photo polymerizable material is being poured onto the object just formed, again while forming a meniscus. The mold is again closed and irradiated. This time the photomask is the stencil for the intended surface structure of the product. This may for example be a pattern of shallow dimples or narrow ribs which are to improve the adherence of growing tissue after implantation. It may also be in the form of numbers, letters, meshes, shapes corresponding to the embossed pattern on the surface of the formed product Alternately the first photomask could be used as well in the second step.

After the mold has been opened, excess unhardened photocurable material is being removed by dissolving it in a suitable solvent. In this step the final shape of the product is determined. Suitable solvents include without being limited to, lower alcohols like ethanol, methanol, propanol, i-propanol, ketones like propanone, 4-methyl-pentan-2-one and butanone as well as mixture s of these.

In order to remove all residual monomers which are left in the cured material and could irritate surrounding tissue after implantation, a final UV exposure is now done, followed by soaking the product in hot water of 90 to 100° C. for at least 30 min.

Up to now the product was still adhered to the mold base. It is now separated from the base and placed in the closed container with Isopropanol at the temperature of between −22 and +12° C. for 3 to 24 hours. After vacuum drying the product is ready.

The formulation of the photocurable material is based upon acrylates and is as follows:

The first photo-curable material is a composition comprising
25-40 wt.-% benzyl methacrylate
50-70 wt.-% oligourethane methacrylate
1-5 wt.-% methacrylic acid
1-5 wt.-% octyl methacrylate.

The second photo-curable material is a composition comprising
20-30 wt.-% phenoxyethyl methacrylate
20-30 wt.-% oligourethane methacrylate 1000F
35-45-wt.-% oligourethane methacrylate 5000F
1-5 wt.-% methacrylic acid
1-5 wt.-% ethylene glycol monomethacrylate.

The optimal composition has to be determined by pretests.

The formulation advantageously contains other ingredients which are common in the field of photocurable materials. These are for example effective amounts of additives capable of initiation of radical polymerization, optical sensitization and/or inhibiting thermal polymerization, dyes or pigments, stabilizers, and the like. Examples are 3, 5-di-t-butyl-o-quinone, azo-bis-isobutyronitrile, 3, 5-di-t-butyl-o-quinone and/or 2, 2-dimethoxyphenylacetophenone. [0022] The invention will be further explained by means of the accompanying drawings, which show specific embodiments of the mold used.

An exemplary mold consists of a base, a cover, and the limiting ring. The upper part of the mold is equipped with a photomask, which is protected by PET-film. A photocurable material is poured onto the base and forms the meniscus on its upper surface.

To manufacture elastic artificial implants for restorative and reconstructive surgery, a casting mold is used consisting of two parts made e. g. of optically transparent material such as glass.

Onto the lower part of the mold the first photo-curable material is poured, consisting of:
benzyl methacrylate—31.68 wt. %;
methacrylic acid—1.97 wt. %;
octyl methacrylate—1.97 wt. %;
dinitrilazo-bis-isobutyric acid—0.005 wt. %;
2, 2-dimethoxy-phenylacetophenone—0.88 wt. %;
3, 5-di-t-butyl-o-quinone—0.01 wt. %;
inorganic pigment ultramarine 463—1.0 wt. %;
oligourethane methacrylate—the rest,
   with the formation of the upper meniscus. The base is covered with the upper part of the mold, in which the limiting ring and the photo-mask corresponding to the outer geometrical dimensions of the product to be formed and protected by the PET-film are fixed to the cover. The two parts of the mold are firmly pressed together and irradiated with UV light, the wave length being 360-380 nm, all over the entire surface of the upper part of the mold. The irradiation time is determined empirically so that the curing of the photosensitive composition takes place all the way through the depth of the layer. Then the parts of the mold are separated and onto the lower part of the mold with the layer that has just been formed, the second liquid photosensitive material is poured forming the meniscus, the composition of the second material being:
oligourethane methacrylate 1000F—25.8 wt. %,
phenoxyethyl methacrylate—25.6 wt. %,
methacrylic acid—4.46 wt. %,
mono methacrylic ethylene glycol ether—4.46 wt. %,
dinitrilazo-bis-isobutyric acid—0.005 wt. %,
2, 2-dimethoxy-phenylacetophenone—0.775 wt. %,
3, 5-di-t-butyl-o-quinone—0.01 wt. %,
oligourethane methacrylate 5000F—the rest.

The base is covered with the upper part of the mold on which are fixed the limiting ring and the photo-mask having transparent and opaque areas in the form of numbers, letters, meshes, shapes corresponding to the embossed pattern formed on the surface of the product, protected by PET-film. The two parts of the mold are then firmly pressed together and the mold is irradiated all over the entire surface of the upper part of the mold. Then the mold parts are separated. The product stays on the base of the mold with the remnants of the uncured liquid material that during the time of irradiation was under the opaque areas of the photo-mask. The product is carefully developed in a suitable solvent such as isopropyl alcohol, then the resulting product, without separating it from the mold, is additionally irradiated with UV light for 3-10 minutes in bi-distilled water at T=40-60° C. Then the mold is additionally placed into a container with bi-distilled water and is soaked for 30-45 minutes at a constant T=100° C. Next, the product is separated from the mold and placed in a closed container with isopropyl alcohol for 3-24 hours at the temperature of −20 C to +12° C., after which the product undergoes a thermal vacuum drying at 40-70° C. for 1-6 hours.

This way it is possible to make implants that have two types of surfaces different in structure (FIG. 5): a smooth lower surface, which was in contact with the surface of the base, and a structured surface created by irradiation through the corresponding photomask. This letter surface will after implantation grow together with the adjacent tissue. This smooth surface will not grow together with the tissue and will remain movable. Thus generation of stress around the implant is avoided. In another embodiment of the method of the invention it is possible to provide both surfaces with structure or even both surfaces without the surface structure.

The following working examples and application tests are a further illustration of the method of the invention:

Example 1. To manufacture elastic artificial implants for restorative and reconstructive surgery a casting mold is used consisting of two parts made of glass (FIG. 1).

Onto the lower part 2 of the mold a first light-sensitive material 4 (number 1) is poured, consisting of:

| | |
|---|---|
| benzyl methacrylate | 31.68 wt. %; |
| methacrylic acid | 1.97 wt. %; |
| octyl methacrylate | 1.97 wt. %; |
| dinitrilazo-bis-isobutyric acid | 0.005 wt. %; |
| 2,2-dimethoxy-phenylacetophenone | 0.88 wt. %; |
| 3,5-di-t-butyl-o-quinone | 0.01 wt. %; |
| inorganic pigment ultramarine 463 | 1.0 wt. %; |
| oligourethane methacrylate | the rest, |

whereby an upper meniscus is formed (FIG. 2). The lower part of the mold with the first light sensitive material is covered with the upper part of the mold on which are fixed the limiting ring 3 and the photo-mask 5, which corresponds to the outer geometrical dimensions of the product to be formed and which is protected by the PET-film 6 (FIG. 3). The two parts of the mold are firmly pressed together and irradiated with UV light of a wave length between •=360-380 nm, all over the entire surface of the upper part of the mold. The irradiation time is determined empirically so that the curing of the photosensitive composition takes place all the way through the depth of the layer. Then the parts of the mold are separated and onto the lower part of the mold with a layer that has just been formed, a second liquid photosensitive material number 2 is poured together with the meniscus. This second material has the following composition:

| | |
|---|---|
| oligourethane methacrylate 1000F | 25.8 wt. %; |
| phenoxyethyl methacrylate | 25.6 wt. %; |
| methacrylic acid | 4.46 wt. %; |
| mono methacrylic ethilene glycol ether | 4.46 wt. %; |
| dinitrilazo-bis-isobutyric acid | 0.005 wt. %; |
| 2,2-dimethoxy-phenylacetophenone | 0.775 wt. %; |
| 3,5-di-t-butyl-o-quinone | 0.01 wt. %; |
| oligourethane methacrylate 5000F | the rest. |

The lower part 2 of the mold is covered with the upper path on which are fixed the limiting ring 3 and the photo-mask 5 (FIG. 4) having transparent and opaque areas in the form of numbers, letters, meshes, shapes corresponding to the embossed pattern formed on the surface of the product to be formed, and which is protected by PET-film. The two parts of the mold are then firmly pressed together and the mold is irradiated all over the entire surface of the upper part of the mold. Then the mold parts are separated. The product stays on the lower part of the mold with the remnants of the uncured liquid material that during the time of irradiation was under the opaque areas of the photo-mask. The product is carefully developed in isopropyl alcohol, then the resulting product, without separating it from the mold, is additionally irradiated with UV light for 3-10 min. in bidistilled water at T=40-60° C. Then the mold with the product is again placed into a container with bidistilled water and is soaked for 30-45 minutes at a constant T=100° C. Next, the product is separated from the mold and placed in a closed container with isopropyl alcohol for 3−24 hours at the temperature of −20 C to +12° C., after which the product undergoes a thermal vacuum drying at 40-70° C. for 1-6 hours. In this way an implant is produced, that has two types of surfaces different in structure (FIG. 5), so the implant does not move on its one side and can move freely on its other side, sliding on the tissues. Such an implant can be used, for instance, in neurosurgery of the brain or spinal cord to reduce the trauma of the tissues and to provide for an a reactive postoperative period.

Patient Z., female, born 1947, admitted to hospital 25 Jan. 2008 MLPU “City Clinical Hospital No 39” of the city of Nizhny Novgorod, with a diagnosis of meningeoma in the left frontal region. 29 Jan. 2008 the patient underwent resection craniotomy, the meningeoma was removed. As a result of the removal of the tumor originating from the dura mater, a 3×3 cm defect of the dura mater was formed. The plasty of the defect was performed using plastic implants for the dura mater plastic defects. The postoperative period went without complications. 13 Feb. 2008 the patient was discharged to outpatient treatment.

Example 2. An artificial elastic implant for restorative and reconstructive surgery is made as in Example 1, but, before the developing takes place, onto the lower part of the mold with the layer that has just been formed, the liquid photosensitive material number 1 is poured to form a meniscus. In this way an implant is produced, which has surfaces different in structure but identical in elasticity (FIG. 5); such an implant can be used, for instance, for complicated neurosurgical interventions on the brain—in case of swelling or dislocation to reduce the trauma of tissues and to provide for a reactivity in the post-operative period.

Patient K., male, age 43 was hit by a car Oct. 4, 2008 and admitted to MLPU “City Clinical Hospital No 39.” The MR-tomograms of the patient revealed an acute subdural hematoma in the right fronto-temporo-parietal region, causing a 4 mm dislocation of the brain to the left. Oct. 5, 2008 the patient underwent resection craniotomy in the right temporo-parietal region, and the removal of acute subdural hematoma. After the removal of the subdural hematoma, the brain spread out into the burr window, which formed a TMO defect. Plasty using the implant in question was performed. In the immediate postoperative period the patient's condition slightly improved: the restoration of consciousness to a deep stunning. But 8 days later the patient re-booted into the 1st stage coma. MR-tomography was done again. It revealed a delayed injury—a bruise and crush of the left temporal lobe, causing dislocation of midline structures to the right by 3 mm. Oct. 13, 2008 the patient was subjected to decompressive craniotomy in the left temporo-parietal region, removing the source of injury—a bruise and crush of the left temporal lobe. Plasty of TMO using the implant in question was performed as well. The postoperative period was uneventful. The patient's condition gradually improved and on November 21 in a satisfactory condition he was discharged for outpatient treatment to a neurologist. In the neurological status moderate cognitive and mnestic violations were retained. Feb. 10, 2009 the patient was re-hospitalized for cranioplasty. February 14th the patient underwent Xeno-cranioplasty in both temporo-parietal regions. It should be noted that between the brain, the implant and the overlying soft tissues no scar adhesions had formed, due to which the surgery duration was decreased.

Example 3. An artificial elastic implant for restorative and reconstructive surgery is made as in Example 1, but, before superimposing the upper part of the mold on the lower part of the mold, the liquid photosensitive material number 2 is poured to form the meniscus. This way we receive an implant that has surfaces different in structure but identical in elasticity (FIG. 5): parietal that is intended for contacting with the abdominal wall, and visceral that is intended for contacting with the abdominal cavity, which allows to use it, for example, for reconstructive surgery of the abdominal wall by the intra-abdominal (intraperitoneal) plasty, to reduce the trauma of tissues and to provide for a reactivity in the post-operative period.

Example 4. An artificial elastic implant for restorative and reconstructive surgery is made as in Example 1, but, before superimposing the upper part of the mold on the lower part of the mold, liquid photosensitive material number 1 is poured together with the meniscus, then it is covered with the upper part of the mold on which are fixed the limiting ring and the photo-mask having transparent and opaque areas in the form of numbers, letters, meshes, shapes corresponding to the embossed pattern formed on the surface of the product, protected by PET-film, the two parts of the mold firmly pressed together, irradiated all over the entire surface of the upper part of the mold. This way we get an implant having a surface structure that could be penetrated by the connective tissues of the body; this implant can be used, for example, in the surgery of inguinal hernias according to the method of Lichtenstein, to reduce the trauma of tissues and to provide for a reactivity in the post-operative period.

Patient S., male, age 52, admitted to MLPU “City Hospital No 35” 28 Oct. 2007 by emergency service. He was brought in by an emergency team with complaints of severe pain in the right inguinal region, repeated vomiting, the presence of a painful protrusion of the right groin. On examination, he was diagnosed with incarcerated inguinal-scrotal hernia on the right. Based on these emergency indications a surgery was performed—herniotomy using the above-described implant. Smooth post-operative period. Healing by first intention. Suppuration, seromas, infiltrates and fistula were not noted. Discharged in satisfactory condition on day 7. Examined in six weeks. The plasty zone was consistent. No signs of relapse of hernia. An ultrasound scan of the implantation area revealed no liquid formation. The implant was without signs of deformation or dislocation.

Example 5. An artificial elastic implant for restorative and reconstructive surgery is made as in Example 1, but, before superimposing the upper part of the mold on the lower part of the mold, liquid photosensitive material number 2 is poured to form a meniscus, then it is covered with the upper part of the mold on which are fixed the limiting ring and the photo-mask having transparent and opaque areas in the form of numbers, letters, meshes, shapes corresponding to the embossed pattern formed on the surface of the product, protected by PIT-film, the two parts of the mold firmly pressed together, irradiated all over the entire surface of the upper part of the mold. This way we get an implant having a surface structure that could be penetrated by the connective tissues of the body; this implant can be used, for example, in the surgery of inguinal hernias according to the method of Trabucco, to reduce the trauma of tissues and to provide for a reactivity in the post-operative period.

Patient B., male, age 57, was admitted on an emergency basis with severe pain in the left inguinal region. He reported that he had had a bilging in this area for many years, which of yesterday stopped going back into the abdomen and became acutely painful. When examined at MLPU “City Hospital No 35,” 28 Nov. 2007, he was diagnosed with incarcerated inguinal-scrotal hernia on the left. Based on these emergency indications a surgery was performed—herniotomy using the above-described implant. Postoperative period went without complications. Seromas, suppuration, infiltration in the area of operations was not observed. The wound healed by first intention. Discharged in a satisfactory condition on day 6.

Example 6. An artificial elastic implant for restorative and reconstructive surgery is made as in Example 1, but after additional irradiation with UV light, the mold is additionally placed into a container of bi-distilled water at constant T=20° C. to soak for 30-45 minutes. Preclinical toxicity study of aqueous extract of the implant according to GOST R ISO 10993-11-2009 by ultraviolet spectroscopy showed the exceeding of the allowable values by 0.2 OP units (the maximum allowed OP value of the aqueous extract is 0.15). No clinical studies were conducted.

Example 7. An artificial elastic implant for restorative and reconstructive surgery is made as in Example 1, but after additional irradiation with UV light, the mold is additionally is placed into a container of bi-distilled water at constant T=100° C. to soak for 3 minutes. Preclinical toxicity study of aqueous extract of the implant according to GOST R ISO 10993-11-2009 by ultraviolet spectroscopy showed the exceeding of the allowable values by 0.12 OP units (the maximum allowed OP value of the aqueous extract is 0.15). No clinical studies were conducted.

In an example where the parameters of the method of manufacturing the implant correspond to the invention formula, the implants have high elasticity, a minimal impact on the surrounding organs and tissues, have both the same types of surfaces and the surfaces that vary in texture and smoothness, are of high biological stability, provide for a reactivity in the post-operative period. Deviations from the method that strictly follows the invention formula lead to the formation of the implant that does not have a low enough toxicity, which can have negative effects on living tissue.

Although the present disclosure includes certain embodiments, examples and applications, it will be understood by those skilled in the art that the present disclosure extends beyond the specifically disclosed embodiments to other alternative embodiments and/or uses and obvious modifications and equivalents thereof, including embodiments which do not provide all of the features and advantages set forth herein. For example, while FIGS. 1-6 depict embodiments that have square or circular shapes, implants may have any other suitable shape. Accordingly, the scope of the present disclosure is not intended to be limited by the specific disclosures of preferred embodiments herein, and may be defined by claims as presented herein or as presented in the future.

1. A surgical implant for reconstruction of soft tissues comprising:
an elastic film formed from a non-porous biologically compatible cross-linked copolymer based on 50-75 percent by weight multi-functional urethane (meth)acrylate oligomers and 20-50 percent by weight methacrylate monomers; the elastic film having:
a first smooth anti-adhesive surface, and
a second relief surface opposite the first surface, the second surface having indentations, and
a reinforcement element in a form of a woven synthetic mesh enclosed inside the elastic film,
wherein the reinforcement element is covered with the elastic film across an entire first smooth anti-adhesive surface area of the implant and
wherein portions of the reinforcement element are exposed through the indentations of the second relief surface.

2. The surgical implant of claim 1, wherein the first smooth surface has a roughness that does not exceed about 50 nanometers.

3. The surgical implant of claim 1, wherein the reinforcement element comprises polypropylene mesh.

4. The surgical implant of claim 1, wherein the reinforcement element comprises polyester mesh.

5. The surgical implant of claim 1, wherein the elastic film has the oval shape.

6. The surgical implant of claim 1, wherein the elastic film has the shape of a rectangle with rounded corners.

7. The surgical implant of claim 1, wherein the elastic film is continuous without holes or apertures such that the reinforcement element is covered with the film across the entire first smooth anti-adhesive surface area of the implant.

8. The surgical implant of claim 1, wherein the elastic film comprises holes through the film in an area of the indentations of the second relief surface.

9. The surgical implant of claim 1, wherein the elastic film comprises an inscription for indication of film surfaces.

10. The surgical implant of claim 1, wherein the implant comprises openings with pre-installed polypropylene filaments for fixation of the implant.

11. The surgical implant of claim 1, wherein the cross-linked copolymer comprises 50-70 weight percent of oligourethane methacrylate and 1-5 weight percent of octyl methacrylate and 25-40 weight percent of benzyl methacrylate.

12. The surgical implant of claim 1, wherein the elastic film is processed by soaking in suitable solvent selected from: methanol, propanol, i-propanol, propanone, water or the mixture of them.
